(19) United States
(12) Patent Application Publication
(10) Pub. No.: US 2021/0217501 A1
(43) Pub. Date: Jul. 15, 2021

(54) LEARNING DEVICE AND LEARNING METHOD

(71) Applicant: FUJITSU LIMITED, Kawasaki-shi (JP)

(72) Inventor: Kyoumoto MATSUSHITA, Kawasaki (JP)

(73) Assignee: FUJITSU LIMITED, Kawasaki-shi (JP)

(21) Appl. No.: 17/130,519

(22) Filed: Dec. 22, 2020

(30) Foreign Application Priority Data
Jan. 10, 2020 (JP) 2020-002961

(51) Int. Cl. G16C 20/70; G16C 20/20; G06N 20/00

(52) U.S. Cl. CPC G16C 20/70; G06N 20/00; G16C 20/20

(57) ABSTRACT

A non-transitory computer-readable recording medium having stored therein a learning program that causes a computer to execute a process, the process includes inputting a first compound name into a model that outputs a character string that represents a chemical structure in response to an input of a compound name, acquiring a result output from the model in response to an input of the first compound name, and executing machine learning of the model, based on a cross entropy error determined based on the result and the character string that represents the chemical structure of a first compound indicated by the first compound name, and a difference in a number of atoms determined based on the result and the number of atoms of each element that forms the first compound.

### CROSS-REFERENCE TO RELATED APPLICATION

This application is based upon and claims the benefit of the prior Japanese Patent Application No. 2020-002961, filed on Jan. 10, 2020, the entire contents of which are incorporated herein by reference.

### FIELD

The embodiments discussed herein are related to a learning device and learning method.

### BACKGROUND

A rule-based method may be considered as a method for creating a chemical structure based on a compound name.

FIG. 6 is a diagram illustrating a method of predicting a chemical structure based on a rule base. In the rule base method, a compound name is divided into a plurality of words, and then each word is replaced with a partial chemical structure by referring to a dictionary or the like. Then, a chemical structure corresponding to the compound name is predicted by combining the plurality of created partial chemical structures.

Related techniques are disclosed in, for example, Japanese Laid-Open Patent Publication No. 2019-128603, and International Publication Pamphlet No. WO 2019/048965.

### SUMMARY

According to an aspect of the embodiments, a non-transitory computer-readable recording medium having stored therein a learning program that causes a computer to execute a process, the process includes inputting a first compound name into a model that outputs a character string that represents a chemical structure in response to an input of a compound name, acquiring a result output from the model in response to an input of the first compound name, and executing machine learning of the model, based on a cross entropy error determined based on the result and the character string that represents the chemical structure of a first compound indicated by the first compound name, and a difference in a number of atoms determined based on the result and the number of atoms of each element that forms the first compound.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating a hardware configuration of a computer system as an example of an embodiment;

FIG. 2 is a diagram illustrating a functional configuration of the computer system as an example of the embodiment;

FIG. 3 is a diagram showing an outline of a neural network;

FIG. 4 is a diagram illustrating an error in the number of atoms in the computer system as an example of the embodiment;

FIG. 5 is a diagram illustrating a process by a deep learning processing unit of the computer system as an example of the embodiment;

FIG. 6 is a diagram illustrating a method of creating a chemical structure by a rule base;

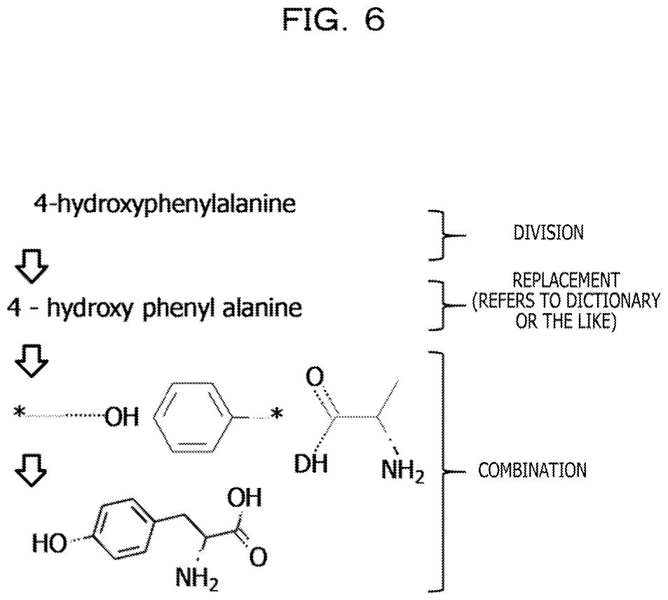

FIG. 7 is a diagram illustrating a method of creating a chemical structure by a statistical method using a neural network; and

FIG. 8 is a diagram illustrating the chemical structure created by the statistical method using the neural network.

### DESCRIPTION OF EMBODIMENTS

In the rule base method, when dividing the compound name and replacing the compound name with the chemical structure, a partial chemical structure that is not in the vocabulary or a partial chemical structure that does not follow the rule may not be processed. Although systematic nomendatures for describing compound names, such as International Union of Pure and Applied Chemistry (IUPAC) nomendature, are publidy known, not all of the compound names are correctly described according to such nomendatures. Therefore, the rule base method does not work when there is a vocabulary or law that is not described in the rule.

In addition, as a method of creating a chemical structure from a compound name, a statistical method of analyzing a causal relationship inherent in data by using translation by a neural network is also conceivable. For example, a method using a transformer or a Long Short Term Memory (LSTM) may be used.

A neural network refers to a computer scientific architecture that models a biological brain. In recent years, with the development of neural network technology, a research has been actively conducted on various electronic systems by utilizing neural networks to analyze input data and extract effective information.

However, such a statistical method lacks a system that place importance on accuracy, such as chemical structure, because research is actively conducted in the field of translation of natural languages where there is no fixed answer. Therefore, although similar compounds are produced, they may not match exactly, such as an occurring of the incorrect numbers of atoms.

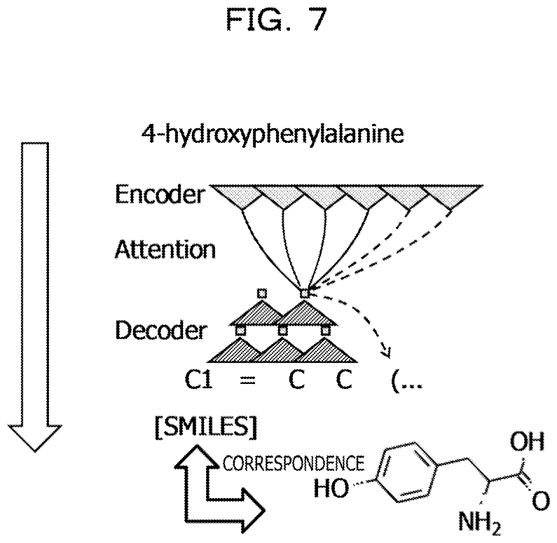

FIG. 7 is a diagram illustrating a method of creating a chemical structure by a statistical method using a neural network. In the example illustrated in FIG. 7, after encoding a compound name, the attention (self-attention) for all combinations of encoded words is calculated to obtain an intermediate representation, and then decoding is performed to generate characters. After that, the characters are associated with a chemical structure represented by Simplified Molecular Input Line entry System (SMILES) notation to create the chemical structure.

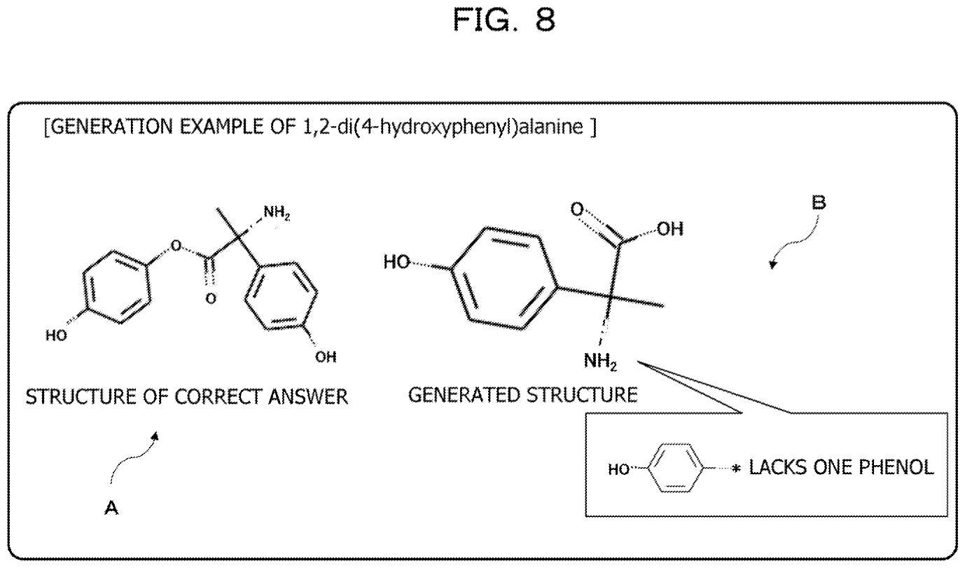

FIG. 8 is a diagram illustrating a chemical structure created by a statistical method using a neural network. FIG. 8 illustrates a chemical structure (see symbol B in FIG. 8) created by a statistical method using correct answer data (see symbol A in FIG. 8) and a neural network for a chemical structure of a compound name “1,2-di(4-hydroxyphenyl)alanine.”

That is, in the example illustrated in FIG. 8, the chemical structure (see symbol B) created as the chemical structure of the compound name “1,2-di(4-hydroxyphenyl)alanine” lacks one phenol with respect to the correct answer structure (see symbol A). In this manner, in the statistical method using the neural network, an error in the number of atoms may occur when a chemical structure is generated.

Hereinafter, an embodiment of the technology for suppressing an error in the number of atoms when generating a chemical structure based on a compound name will be described with reference to the accompanying drawings. However, the following embodiment is merely an example and is not intended to exclude the application of various modifications and techniques not explicitly described in the embodiment. That is, the present embodiment may be variously modified and implemented without departing from the spirit thereof. Further, each figure is not intended to include only the constituent elements shown in the figure, but may include other functions and the like.

[Configuration]

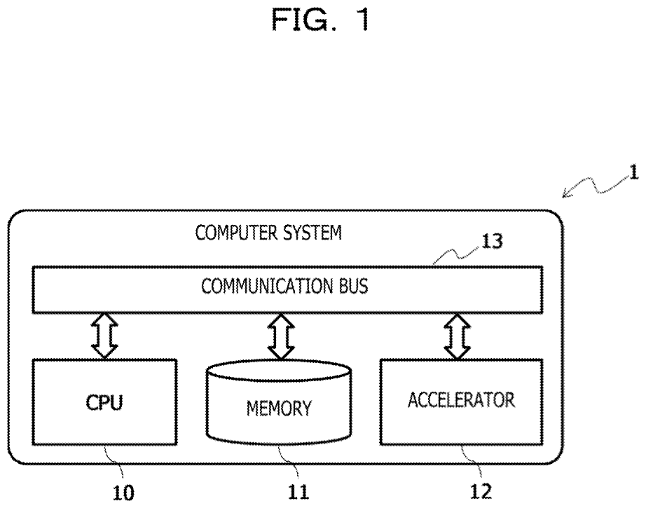

FIG. 1 is a diagram illustrating a hardware configuration of a computer system 1 as an example of the embodiment.

The computer system 1 is an information processing device and implements a neural network. As illustrated in FIG. 1, the computer system 1 includes a Central Processing Unit (CPU) 10, a memory 11, and an accelerator 12. The CPU 10, the memory 11, and the accelerator 12 are communicably connected to each other via a communication bus 13. A data communication is performed within the computer system 1 via the communication bus 13.

The memory 11 is a storage memory including a Read Only Memory (ROM) and a Random Access Memory (RAM). A program executed by the CPU to be described later and data for this program are written in the ROM of the memory 11. A software program on the memory 11 is appropriately read and executed by the CPU 10. The RAM of the memory 11 is used as a primary storage memory or a working memory. Parameters used for quantization of weights and the like are also stored in the RAM of the memory 11.

The accelerator 12 executes arithmetic processing necessary for calculation of the neural network, such as matrix operation.

The CPU 10 is a processing device (a processor) that performs various controls and calculations, and controls the entire computer system 1 based on an installed program. Then, the CPU 10 executes a deep learning processing program (learning program (not shown)) stored in the memory 11 or the like to implement a function as a deep learning processing unit 100 to be described later.

In addition, the deep learning processing program may be configured to include a chemical structure creation processing program (estimation program). The CPU 10 executes a chemical structure creation processing program (not shown) stored in the memory 11 or the like to implement a function as a chemical structure creation processing unit 101 to be described later.

Then, the CPU 10 of the computer system 1 functions as the deep learning processing unit 100 by executing the deep learning processing program (learning program or estimation program). The computer system 1 functions as a learning device by executing the deep learning processing program (learning program) and functions as an estimation device by executing the chemical structure creation processing program (estimation program).

In addition, a program (learning program or estimation program) for implementing the function of the deep learning processing unit 100 is provided in a form recorded on a computer-readable recording medium such as a flexible disk, a CD (CD-ROM, CD-R, CD-RW, etc.), a DVD (DVD-ROM, DVD-RAM, DVD-R, DVD+R, DVD-RW, DVD+RW, HD DVD, etc.), Blu-ray disc, magnetic disc, optical disc, magneto-optical disc, or the like. Then, the computer (computer system 1) reads the program from the recording medium, transfers the program to an internal storage device or an external storage device, and stores the program for use. Alternatively, the program may be recorded in a storage device (recording medium) such as a magnetic disc, an optical disc, a magneto-optical disc, or the like and provided from the storage device to the computer via a communication path.

When implementing the function of the deep learning processing unit 100, the program stored in the internal storage device (RAM or ROM of the memory 11 in this embodiment) is executed by a microprocessor (CPU 10 in this embodiment) of the computer. At this time, the computer may read and execute the program recorded in the recording medium.

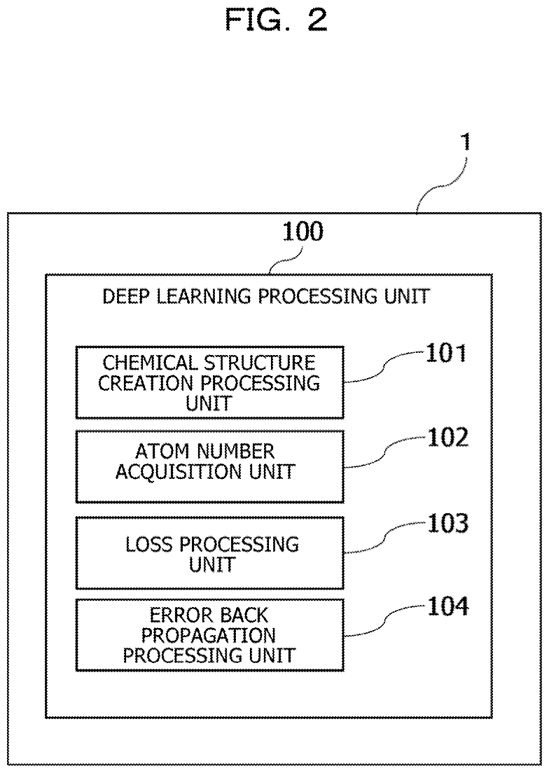

FIG. 2 is a diagram illustrating a functional configuration of the computer system 1 as an example of the embodiment.

The computer system 1 has a function as a deep learning processing unit 100, as illustrated in FIG. 2. The deep learning processing unit 100 carries out deep learning in a neural network. As illustrated in FIG. 2, the deep learning processing unit 100 includes a chemical structure creation processing unit 101, an atom number acquisition unit 102, a loss processing unit 103, and an error back propagation processing unit 104.

The chemical structure creation processing unit 101, using translation by a neural network, creates (estimates) a chemical structure from a compound name using a method of analyzing a causal relationship inherent in data by a statistical method. The chemical structure creation processing unit 101 has a function as a model (estimation model) that outputs a character string representing a chemical structure in response to the input of a compound name, and inputs an input processing target compound name to this estimation model.

The estimation model is created by machine learning that has been executed using a loss function based on a difference between the number of atoms of each element included in the chemical structure created (estimated) by the chemical structure creation processing unit 101 and the number of atoms of each element included in the correct answer data of the chemical structure (correct answer chemical structure).

The neural network may be a hardware circuit, or may be a virtual network formed by software that connects the layers that are virtually constructed on a computer program by the CPU 10 or the like.

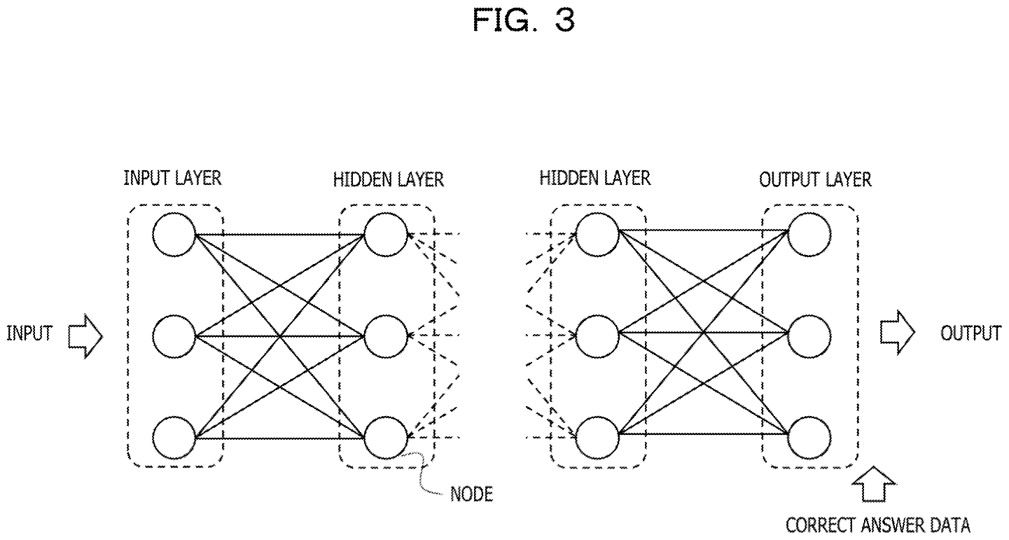

FIG. 3 illustrates the outline of the neural network. The neural network illustrated in FIG. 3 is a deep neural network including a plurality of hidden layers between an input layer and an output layer. Each hidden layer is, for example, a convolutional layer, a pooling layer, or a fully connected layer. In FIG. 3, circles shown in each layer indicate nodes that execute predetermined calculations.

The neural network executes a process in the forward direction (forward propagation process) that transmits information obtained by calculation sequentially from the input side to the output side, for example, by inputting input data such as a compound name into the input layer and sequentially executing predetermined calculations in the hidden layer configured by the convolutional layer or the pooling layer. After executing the process in the forward direction, in order to reduce a value of an error function obtained from the output data output from the output layer and the correct answer data, a process in the backward direction (back propagation process) that determines a parameter used in the process in the forward direction is executed. Then, an update process for updating a variable such as a weight or the like is executed based on a result of the back propagation process.

The chemical structure creation processing unit 101 creates (estimates) a chemical structure indicated by a compound name (first compound) of a processing target by executing the process in the forward direction. The compound name of the processing target may be referred to as a processing target compound name.

The chemical structure creation processing unit 101 may create a chemical structure from a compound name using, for example, Transformer or LSTM. The creation of the chemical structure from the compound name may be implemented using a known method, and explanation thereof will be omitted. The chemical structure created by the chemical structure creation processing unit 101 may be represented by, for example, SMILES notation. Hereinafter, the chemical structure created by the chemical structure creation processing unit 101 may be referred to as an estimated chemical structure.

The chemical structure creation processing unit 101 functions as an estimation model that receives an input of a processing target compound name (first compound name) and estimates a character string representing a chemical structure including a plurality of elements according to the input of the compound name. This estimation model is created by machine learning executed using a loss function based on a difference between the number of atoms of each element indicated by the estimation result and the number of atoms of each element indicated by the correct answer data. The chemical structure creation processing unit 101 inputs the received processing target compound name (first compound name) to this estimation model. Then, the chemical structure creation processing unit 101 functions as a processing unit that outputs the character string indicating an estimated chemical structure (first chemical structure) output from the estimation model in response to the input of the processing target compound name, as an estimation result of a chemical structure for the processing target compound name.

The atom number acquisition unit 102 acquires the number of atoms (the atom number) included in the chemical structure (estimated chemical structure). The atom number acquisition unit 102 acquires the number of atoms included in the created chemical structure by counting the number of atoms included in the character string of the estimated chemical structure represented by, for example, the SMILES notation.

The atom number acquisition unit 102 obtains the number of atoms of each element included in the chemical structure based on the character string of the SMILES notation of the chemical structure created by the chemical structure creation processing unit 101. Further, when the chemical structure (correct answer data) of the correct answer of the compound name is input to the deep learning processing unit 100, the atom number acquisition unit 102 similarly obtains the number of atoms of each element included in the chemical structure of the correct answer. The chemical structure of the correct answer of the compound name may be referred to as the correct answer chemical structure.

The loss processing unit 103 calculates a loss that reflects an error of the number of atoms in the chemical structure created by the atom number acquisition unit 102. The loss processing unit 103 functions as an acquisition unit that acquires the result output by the chemical structure creation processing unit 101 in response to the input of the processing target compound name (first compound name).

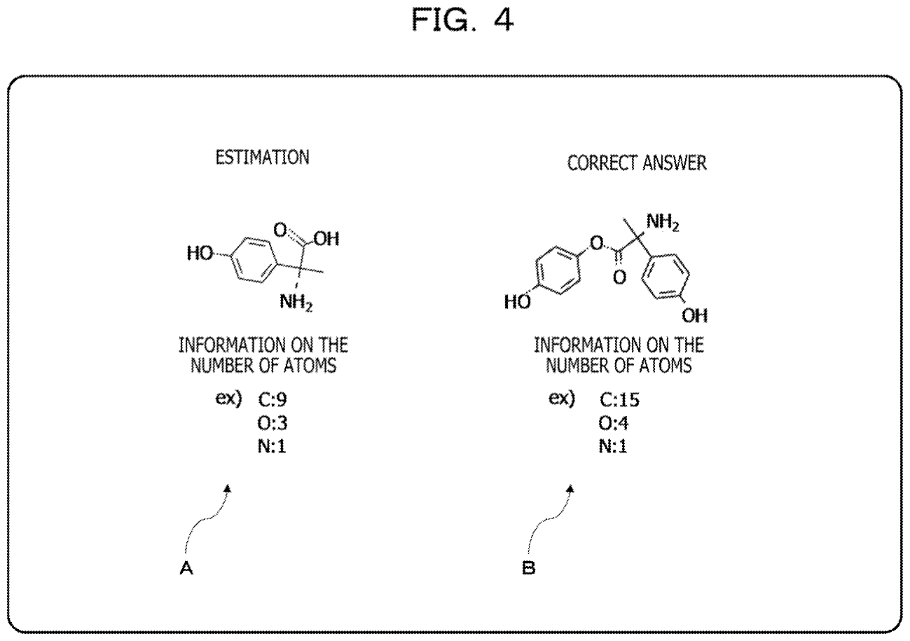

FIG. 4 is a diagram illustrating an error in the number of atoms in the computer system 1 as an example of the embodiment. In FIG. 4, symbol A exemplifies an estimated chemical structure created by the chemical structure creation processing unit 101, and symbol B exemplifies a correct answer chemical structure.

In the example illustrated in FIG. 4, in the estimated chemical structure created by the chemical structure creation processing unit 101, the number of atoms of C is 9, the number of atoms of O is 3, and the number of atoms of N is 1, as indicated by symbol A. Meanwhile, in the correct answer chemical structure, the number of C atoms is 15, the number of O atoms is 4, and the number of N atoms is 1, as indicated by symbol B.

The loss processing unit 103 calculates a loss L by the following equation (1).

$$L = - log p ( t \vert x ) + \frac{λ}{\vert A \vert} {{Σ}_{a ∈ A} ( {N}_{a} ( t ) - {N}_{a} ( y ) )}^{2} \qquad ( 1 )$$

In the above equation (1), “x” is a compound name. The “y” is a value (system output) of SMILES indicating the constituent atoms of the estimated chemical structure created by the chemical structure creation processing unit 101. The “t” is a value (correct answer) of SMILES indicating the constituent atoms of the chemical structure given as the correct answer. The “Na(x)” represents the number of atoms “a” in “x.” The “λ” is a coefficient, which may be appropriately changed and implemented.

In the above equation (1), “−log p(t|x)” is a cross entropy error and is determined based on a character string representing the estimated chemical structure and a character string representing the correct answer chemical structure.

In the above equation (1), an error of the number of atoms is reflected as a loss function (error function) by adding the loss function (error function) in which the error of the number of atoms is expressed as a square error to the cross entropy error as described above.

In order to reduce the value of an error function obtained from the output data output from the output layer and the correct answer data, the error back propagation processing unit 104 executes a process in the backward direction (back propagation process or back propagation) which determines parameters used in a process in the forward direction.

Specifically, the error back propagation processing unit 104 executes a process in the backward direction which determines parameters used in a process in the forward direction so that the loss L calculated according to the above equation (1) by the loss processing unit 103 becomes small. The error back propagation processing unit 104 executes an update process for updating variables such as weights based on a result of the back propagation process. As a result, machine learning is performed with the error in the number of atoms in the estimated chemical structure as a loss.

In the back propagation process, a weight between an intermediate layer and the output layer is corrected based on an error between the output value and the correct answer. Then, based on this corrected value, a weight between the input layer and the intermediate layer is corrected. For example, a gradient descent method may be used as an algorithm for determining the update width of the weight used for the calculation of the back propagation process. The error back propagation processing unit 104 sets a weight or a threshold that minimizes the loss L.

The error back propagation processing unit 104 learns how much the number of atoms of the chemical structure created (predicted) by the chemical structure creation processing unit 101 differs from the number of atoms of the correct answer chemical structure. That is, the error back propagation processing unit 104 combines the number of atoms of each element to be generated with the input compound name.

As described above, the error back propagation processing unit 104 performs a process based on the loss L obtained by the above equation (1). Thus, the error back propagation processing unit 104 implements a machine learning of an estimation model based on a cross entropy error, and a difference in the number of atoms determined based on the number of atoms of each element forming the estimated chemical structure and the number of atoms of each element forming the correct answer chemical structure.

[Operation]

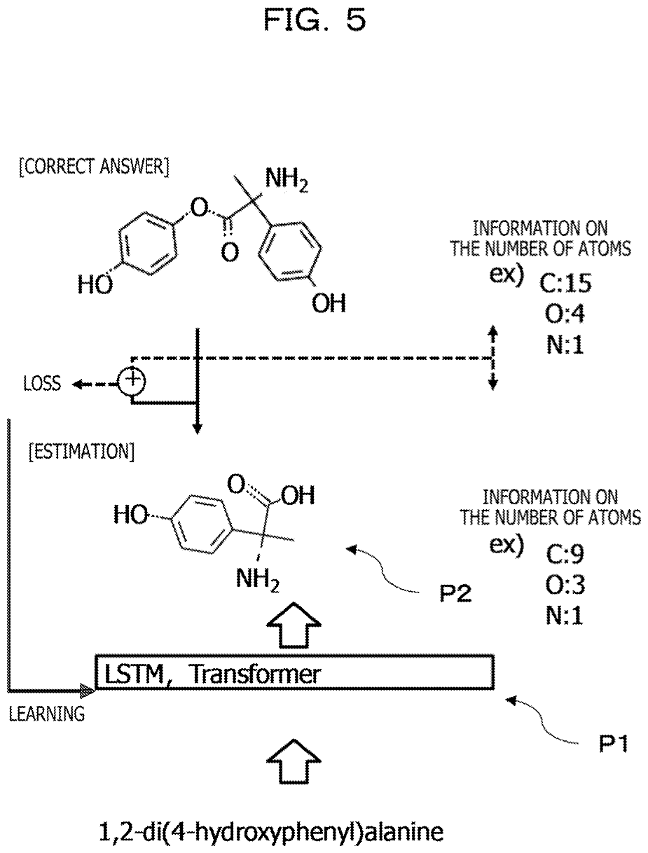

A process (learning step) by the deep learning processing unit 100 of the computer system 1 as an example of the embodiment, which is configured as described above, will be described with reference to FIG. 5.

First, the chemical structure creation processing unit 101 receives an input of a processing target compound name (first compound name). In the example illustrated in FIG. 5, a compound name of “1,2-di(4-hydroxyphenyl)alanine” is input.

The chemical structure creation processing unit 101 uses LSTM, Transformer, or the like to convert the input compound name into a chemical structure using a statistical method (see symbol P1 in FIG. 5). In the chemical structure creation processing unit 101, after the compound name is encoded, the attention (self-attention) for all combinations of the encoded words is calculated to obtain an intermediate representation, and then decoding is performed to generate a character.

Subsequently, the chemical structure creation processing unit 101 creates (estimates) a chemical structure by associating the character with a chemical structure represented by the SMILES notation (see symbol P2 in FIG. 5). The chemical structure creation processing unit 101 outputs the character string indicating the created chemical structure to a predetermined storage area such as the memory 11.

The atom number acquisition unit 102 acquires, from the memory 11 or the like, a result (estimated chemical structure) output from the chemical structure creation processing unit 101 in response to an input of a processing target compound name. The atom number acquisition unit 102 calculates the number of atoms of each element contained in the chemical structure based on the character string indicating the estimated chemical structure created by the chemical structure creation processing unit 101. When the correct answer chemical structure of the compound name is input to the deep learning processing unit 100, the atom number acquisition unit 102 also calculates the number of atoms of each element included in the correct answer chemical structure.

The loss processing unit 103 calculates a loss L reflecting an error of the number of atoms using the above equation (1). The calculation equation (1) of the loss L reflects a cross entropy error determined based on the character string representing the estimated chemical structure for the processing target compound name and the character string representing the correct answer chemical structure, and a difference between the number of atoms for each element forming the estimated chemical structure and the number of atoms for each element forming the correct answer chemical structure.

The error back propagation processing unit 104 executes a back propagation process that determines parameters used in a process in the forward direction so that the loss L calculated by the loss processing unit 103 becomes small.

That is, the loss processing unit 103 executes a learning of the chemical structure creation processing unit 101 (model) based on the cross entropy error determined based on the character string representing the estimated chemical structure for the processing target compound name and the character string representing the correct answer chemical structure, and the difference between the number of atoms for each element forming the estimated chemical structure and the number of atoms for each element forming the correct answer chemical structure.

Next, a process (use step) of the chemical structure creation processing unit 101 of the computer system 1 as an example of the embodiment configured as described above will be described.

First, the chemical structure creation processing unit 101 receives an input of a processing target compound name (first compound name), and inputs the processing target compound name into a function as an estimation model. The estimation model is generated by the loss processing unit 103 by machine learning executed using a loss function based on a difference between the number of atoms of each element included in the estimated chemical structure created by the chemical structure creation processing unit 101 and the number of atoms of each element included in the correct answer data of the chemical structure (correct answer chemical structure).

The chemical structure creation processing unit 101 outputs a character string indicating the chemical structure (first chemical structure) output from the estimation model in response to an input, as an estimation result of a chemical structure corresponding to an input compound name (first compound name).

[Effects]

As described above, according to the computer system 1 as one embodiment of the present disclosure, the difference in the number of atoms between the created chemical structure and the correct chemical structure is given as a loss during the time of learning of the neural network. As a result, the number of atoms in the chemical structure may be learned so that the number of atoms is correct, thereby improving the reliability.

The loss processing unit 103 reflects the error between the number of atoms of the correct answer chemical structure and the number of atoms of the estimated chemical structure in the above equation (1), as a loss function. As a result, it is possible to learn an error in the number of atoms in the estimated chemical structure, as a loss.

[Others]

The present disclosure is not limited to the above-described embodiment, but various modifications may be carried out without departing from the spirit of the present disclosure.

In addition, in the above-described embodiment, by adding the loss function (error function), in which the error of the number of atoms is expressed as a square error, to the cross entropy error in the equation (1), the error in the number of atoms is added as the loss function (error function). However, the present disclosure is not limited thereto. Other loss functions may be used instead of the cross entropy error. Further, the error of the number of atoms may be applied to a loss function (error function) using a method other than the square error, and various modifications may be implemented. Further, it is possible for those skilled in the art to implement and manufacture the present embodiment based on the above disclosure.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to an illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. A non-transitory computer-readable recording medium having stored therein a learning program that causes a computer to execute a process, the process comprising:
inputting a first compound name into a model that outputs a character string that represents a chemical structure in response to an input of a compound name;
acquiring a result output from the model in response to an input of the first compound name; and
executing machine learning of the model, based on a cross entropy error determined based on the result and the character string that represents the chemical structure of a first compound indicated by the first compound name, and a difference in a number of atoms determined based on the result and the number of atoms of each element that forms the first compound.

2. The non-transitory computer-readable recording medium according to claim 1, wherein the machine learning is executed by using a loss function that includes the cross entropy error, and a square error calculated from the difference in the number of atoms.

3. The non-transitory computer-readable recording medium according to claim 1, wherein the character string that represents the chemical structure is written in SMILES (Simplified Molecular Input Line Entry System) notation.

4. A learning method executed by a processor, the learning method comprising:
inputting a first compound name into a model that outputs a character string that represents a chemical structure in response to an input of a compound name;
acquiring a result output from the model in response to an input of the first compound name; and
executing machine learning of the model, based on a cross entropy error determined based on the result and the character string that represents the chemical structure of a first compound indicated by the first compound name, and a difference in a number of atoms determined based on the result and the number of atoms of each element that forms the first compound.

5. The learning method according to claim 4, wherein the machine learning is executed by using a loss function that includes the cross entropy error, and a square error calculated from the difference in the number of atoms.

6. The learning method according to claim 4, wherein the character string that represents the chemical structure is written in Simplified Molecular Input Line Entry System (SMILES) notation.

7. A learning device comprising:
a memory; and
a processor coupled to the memory and configured to:
input a first compound name into a model that outputs a character string that represents a chemical structure in response to an input of a compound name;
acquire a result output from the model in response to an input of the first compound name; and
execute machine learning of the model, based on a cross entropy error determined based on the result and the character string that represents the chemical structure of a first compound indicated by the first compound name, and a difference in a number of atoms determined based on the result and the number of atoms of each element that forms the first compound.

8. The learning device according to claim 7, wherein the machine learning is executed by using a loss function that includes the cross entropy error, and a square error calculated from the difference in the number of atoms.

9. The learning device according to claim 7, wherein the character string that represents the chemical structure is written in Simplified Molecular Input Line entry System (SMILES) notation.
